(19) United States
(12) Patent Application Publication
(10) Pub. No.: US 2026/0103438 A1
(43) Pub. Date: Apr. 16, 2026

(54) PROBES AND METHODS TO IDENTIFY LIGANDABLE FATTY ACYLATION SITES FOR THERAPEUTIC TARGET IDENTIFICATION

(71) Applicant: The General Hospital Corporation, Boston, MA (US)

(72) Inventors: Xu Wu, Lexington, MA (US); Lu Hu, Malden, MA (US)

(21) Appl. No.: 19/114,987

(22) PCT Filed: Sep. 27, 2023
(86) PCT No.: PCT/US2023/075230

§ 371 Date: Mar. 25, 2025

Related U.S. Application Data
(60) Provisional application No. 63/410,689, filed on Sep. 28, 2022.

(51) Int. Cl. C07C 233/09; C07C 233/05; C07C 311/11; G01N 33/50; G01N 33/68

(52) U.S. Cl. CPC C07C 233/09; C07C 233/05; C07C 311/11; G01N 33/5008; G01N 33/6803

(57) ABSTRACT

The present disclosure relates to compounds of Formula I and methods comprising the use of these compounds to identify ligandable fatty acylation sites.

### CROSS-REFERENCE TO RELATED APPLICATIONS

This application claims the benefit of U.S. Provisional Application No. 63/410,689, filed on Sep. 28, 2022, the entire disclosure of which is incorporated herein by reference in its entirety.

### STATEMENT REGARDING FEDERALLY SPONSORED RESEARCH

This invention was made with government support under grant number R01CA238270 from the National Cancer Institute. The government has certain rights in the invention.

### FIELD

The present disclosure relates to the fields of chemistry and biology. More specifically, to probes and methods to identify hydrophobic pockets in the proximity of reactive nucleophilic residues of proteins, including fatty acylation sites.

### BACKGROUND

Hippo Posttranslational S-palmitoylation modulates protein localization and functions, and has been implicated in neurological, metabolic, infectious diseases, and cancers. Auto-palmitoylation involves reactive cysteine residues on the protein, which directly react with palmitoyl-CoA through thioester transfer reactions. Many structural proteins, transcription factors and adaptor proteins possess “enzyme-like” activities and undergo auto-palmitoylation upon palmitoyl-CoA binding. The present invention provide probes and methods for identifying druggable hydrophobic pockets in proximity to reactive nucleophiles. For instance, the probes and methods described herein are used for identifying druggable hydrophobic pockets of lapidated proteins, including auto-palmitoylated proteins that represent a class of unexplored potential drug targets.

### SUMMARY

Some embodiments provide a compound according to Formula I:

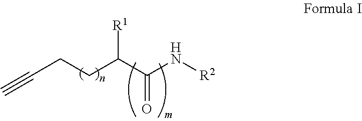

or a pharmaceutically acceptable salt thereof, wherein
   n is 9, 10, 11, 12, 13, or 14;
   R¹ is H or halogen;
   m is 0 or 1; and
   R² is H, —C(═O)—R^{A}, or —S(O)₂—R^{A};
   wherein R^{A} is C₂₋₆ alkenyl or C₁₋₆ haloalkyl.

Some embodiments provide a method for identifying autolipidation, comprising:
(a) contacting a cell, a cellular lysate, or a recombinant protein with a compound described herein forming Complex A;
(b) contacting Complex A with a compound selected from the group consisting of DADPS biotin azide, Dde biotin azide, Dde biotin azide PLUS, Dde biotin picolyl azide, biotin picolyl azide, biotin azide, biotin azide PLUS, and PC azide, forming Complex B;
(c) isolating Complex B; and
(d) identifying the autolipidated protein.

Some embodiments provide a method of identifying a druggable hydrophobic pocket in proximity to reactive nucleophiles of proteins, comprising contacting a cell, a cellular lysate, or a recombinant protein with a compound described herein.

Some embodiments provide a method of identifying ligandable acylation sites comprising contacting a cell, a cellular lysate, or a recombinant protein with a compound described herein.

Some embodiments provide a method of labeling cell lystates or tissue lysates comprising contacting a cellular lysate or a tissue lysate with a compound described herein.

Some embodiments provide a method of identifying auto-palmitoylated proteins comprising contacting a cell, a cellular lysate, or a recombinant protein with a compound described herein.

### DESCRIPTION OF DRAWINGS

FIG. 1 shows TEAD2 labeled with compounds of Formula I in vitro. TEAD2 was treated with compounds of Formula I (1 μM) for 30 mins. The labeling efficiency was characterized by western blot analysis. The inventive probes exhibit superior reactivity. Compound A21, Compound A26, Compound A63, Compound A64, Compound B1, and Compound B4 showed strong labeling efficiency.

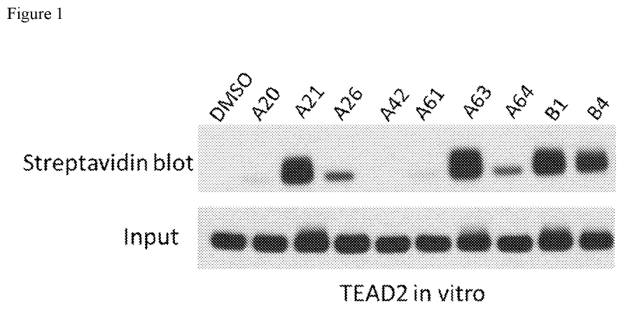

FIG. 2 shows endogenous pan-TEAD labeled with compounds of Formula I in HEK293A cells and lysate. FIG. 2A shows pan-TEAD labeled with compounds of Formula I (10 μM) in HEK293A lysate. FIG. 2B shows pan-TEAD labeled with compounds of Formula I (10 μM) in living cells. The compounds of Formula I showed potent pan-TEAD labeling at a concentration of 10 μM in both cell lysate and living cells.

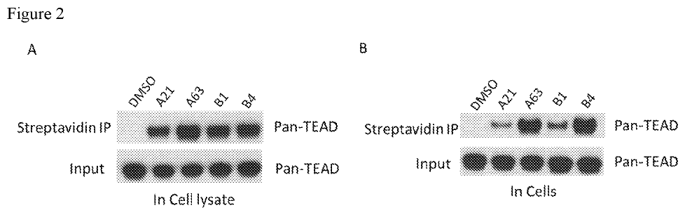

FIG. 3 shows Compound B4 is an activity-based probe for TEAD in cells. HEK293A cells were transfected with Myc-TEAD1 wild type or C344S mutant and treated with 10 M of Compound B4 overnight. Myc-TEAD1 labeled by compound B4 was analyzed by streptavidin pulldown assay and western blot. Mutating cysteine 344 at auto-palmitoylation site inhibited labeling of Myc-TEAD1 by Compound B4.

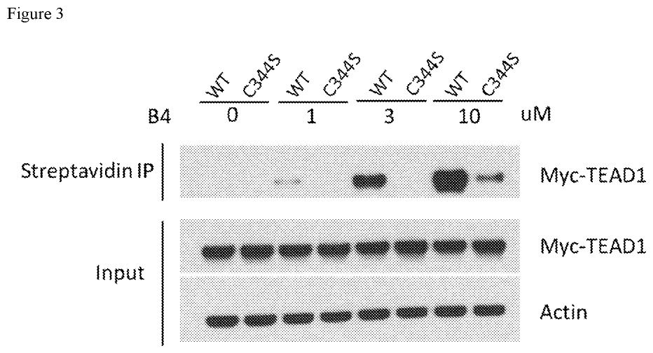

FIG. 4 shows a Venn diagram of shared and unique protein targets of Compound A21, Compound B1, and Compound B4 in HEK293A cells. Mass spectrometry (MS)-based proteomics identified more than 300 proteins that were enriched by covalent probes, which were annotated as auto-palmitoylated candidates. One hundred and sixty proteins are collectively targeted by Compound A21, Compound B1, and Compound B4, while a small portion of proteins are identified as being targeted by only one of Compound A21, Compound B1 and Compound B4.

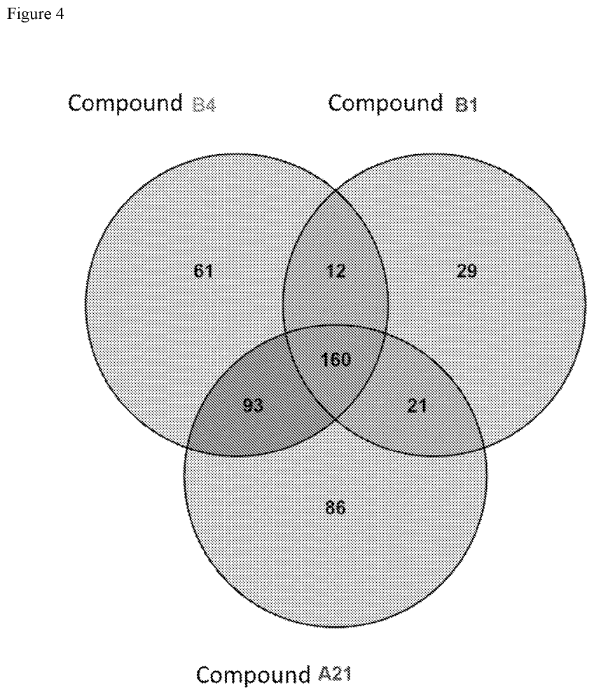

FIG. 5 shows a Venn Diagram that overlaps proteins identified by Compound A21, Compound B1, and Compound B4 with proteins identified using streamlined cysteine activity-based protein profiling (SLC-ABPP), a reported database showing reactivity of cysteines towards covalent fragments. The date showed that 191 proteins enriched by these covalent probes were also identified by SLC-ABPP.

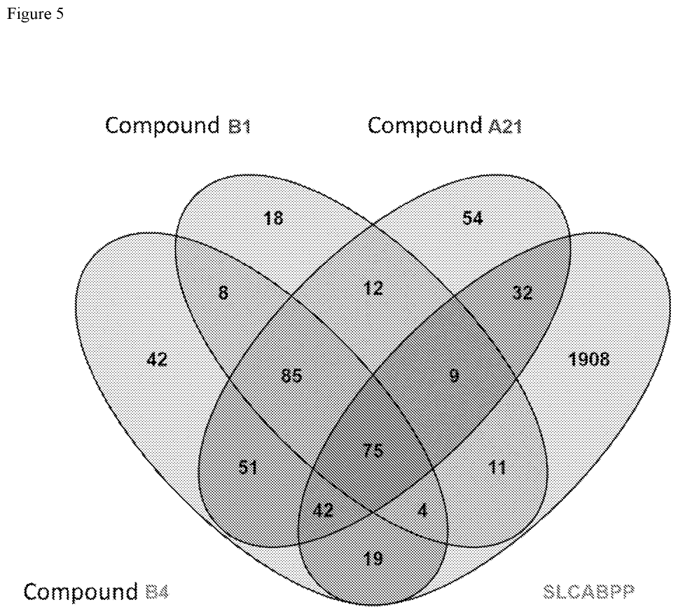

FIG. 6 shows western blot analysis of select proteins from covalent probe-based profiling. Recombinant PCNA (FIG. 6A), RAP1A (FIG. 6B), RRAS2 (FIG. 6C), and IDH1WT/mt (FIG. 6D) were treated with Alkyne Palmitoyl-CoA under indicated concentrations for 30 mins. The labeling efficiency was characterized by western blot analysis. Each of the exemplified proteins are auto-palmitoylated in a dose dependent manner. The somatic mutation version of IDH1, which is frequently detected in many cancers, also shows strong auto-palmitoylation activities.

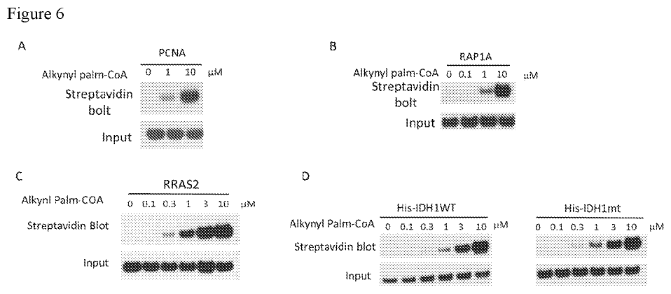

FIG. 7A shows that in vitro labeling of recombinant IDH1 R132H with Compound B4 was suppressed by Compound C20. FIG. 7B shows mutation of cysteine 269 to serine dramatically abolished labeling of IDH1 R132H by Compound B4. FIG. 7C shows LY3410738 (1 μM) inhibited palmitoylation of IDH1mutant (mt), but not IDH1mtC269S. FIG. 7D shows mutation of cysteine 269 to serine blocks enzymatic activities of IDH1mt. Compound C20, a fragment targeting Cys269 of IDH1mt, suppressed Compound B4 labeling in vitro. In addition, only mutating Cysteine 269 to serine in IDH1mt significantly blocked compound B4 labeling. These results collectively demonstrated that compound B4 selectively labeled Cysteine 269 of IDH1mt, which can be targeted by small molecule fragment. Consistently, LY3410738, a IDH1mt inhibitor, also suppressed palmitoylation level of IDH1mt, but not C269S mutant, confirming that compound B4 identifies ligandable site in IDH1 R132H. Mutating Cys269 also suppressed enzymatic activity of IDH1mt.

### DETAILED DESCRIPTION

The public database SwissPalm predicts over 5,000-10,000 putative S-palmitoylated proteins, roughly 10% of the proteome. Recent evidence has linked dysfunctions of protein palmitoylation to various diseases, including cancers and neurological diseases. However, identifying compounds that target protein pockets occupied by lipids or hydrophobic pockets that are in proximity to a nucleophile that is targeted and/or modified by a lipid is challenging. For instance the “druggability” of auto-palmitoylated protein has long been overlooked. Provided herein are a series of chemical probes to detect the potential acyl-binding pocket in proteins. These probes exhibit improved specificity and reactivity, and the probes allow direct labeling fo proteins in cell and in tissue lysates (i.e., without live cells), which greatly expands the utility of the chemical probes. The probes were tested in chemoproteomic studies, and more than 200 putative auto-palmitoylated proteins were identified as new therapeutic targets. In vitro studies reconfirmed that PCNA, RRAS, IDH1 and RAP1A are exemplary auto-palmitoylated proteins.

### Probes

Provided herein is a compound according to Formula I:

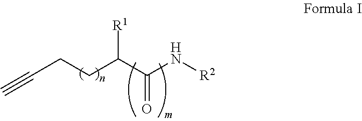

or a pharmaceutically acceptable salt thereof, wherein
   n is 9, 10, 11, 12, 13, or 14;
   R¹ is H or halogen;
   mis 0 or 1; and
   R² is H, —C(═O)—R^{A}, or —S(O)₂—R^{A};
   wherein R^{A} is C₂₋₆ alkenyl or C₁₋₆ haloalkyl.

In some embodiments, when R² is H, then m is 1. In some embodiments, when R² is H, then R¹ is halogen. In some embodiments, when R² is H, then R¹ is Br. In some embodiments, when R² is H, then m is 1 and R¹ is halogen. In some embodiments, when R² is H, then m is 1 and R¹ is Br.

In some embodiments, n is 10, 12, or 14. In some embodiments, n is 10 or 12. In some embodiments, n is 9. In some embodiments, n is 10. In some embodiments, n is 11. In some embodiments, n is 12. In some embodiments, n is 13. In some embodiments, n is 14.

In some embodiments, R¹ is H. In some embodiments, R¹ is halogen. In some embodiments, R¹ is Br, Cl, I, or F. In some embodiments, R¹ is Cl or Br. In some embodiments, R¹ is Br.

In some embodiments, m is 1. In some embodiments, m is 0.

In some embodiments, R¹ is halogen and m is 1. In some embodiments, R¹ is Br and m is 1. In some embodiments, R¹ is H and m is 0.

In some embodiments, R² is H. In some embodiments, R² is C(═O)—R^{A}. In some embodiments, R² is —S(O)₂—R^{A}.

In some embodiments, R^{A} is C₂₋₆ alkenyl. In some embodiments, R^{A} is C₂₋₅ alkenyl. In some embodiments, R^{A} is C₂₋₄ alkenyl. In some embodiments, R^{A} is C₂₋₃ alkenyl. In some embodiments, R^{A} is —CH₂CH₂.

In some embodiments, R^{A} is C₁₋₆ haloalkyl. In some embodiments, R^{A} is C₁₋₅ haloalkyl. In some embodiments, R^{A} is C₁₋₄ haloalkyl. In some embodiments, R^{A} is C₁₋₃ haloalkyl. In some embodiments, R^{A} is C₁₋₂ haloalkyl. In some embodiments, R^{A} is —CH₂Cl, —CH₂I, or —CH₂Br. In some embodiments, R^{A} is —CH₂Cl. In some embodiments, R^{A} is —CH₂I. In some embodiments, R^{A} is —CH₂Br.

In some embodiments, R^{A} is —CH₂Cl, —CH₂I, —CH₂Br, or —CH₂CH₂.

In some embodiments, R² is —C(═O)—CH₂Cl, —C(═O)—CH₂Br, —C(═O)—CH₂I, —C(═O)—CH₂CH₂, or —S(O)₂—CH₂CH₂. In some embodiments, R² is —C(═O)—CH₂Br, —C(═O)—CH₂I, or —S(O)₂—CH₂CH₂. In some embodiments, R² is —C(═O)—CH₂Br or —C(═O)—CH₂I.

In some embodiments,
n is 10 or 12;
R¹ is H;
m is 0; and
R² is —C(═O)—R^{A}, or —S(O)₂—R^{A};
wherein R^{A} is C₁₋₆ alkenyl or C₁₋₆ haloalkyl.

In some embodiments,
n is 10 or 12;
R¹ is H;
m is 0; and
R² is —C(═O)—R^{A}, or —S(O)₂—R^{A};
wherein R^{A} is —CH₂Cl, —CH₂I, —CH₂Br, or —CH₂CH₂.

In some embodiments,
n is 10 or 12;
R¹ is H;
m is 0; and
R² is —C(═O)—CH₂Cl, —C(═O)—CH₂Br, —C(═O)—CH₂I, —C(═O)—CH₂CH₂, or —S(O)₂—CH₂CH₂

In some embodiments,
n is 10 or 12;
R¹ is H;
m is 0; and
R² is —C(═O)—CH₂Br, —C(═O)—CH₂I, or —S(O)₂—CH₂CH₂;

In some embodiments,
n is 10 or 12;
R¹ is H;
m is 0; and
R² is —C(═O)—CH₂Br or —C(═O)—CH₂I.

In some embodiments,
n is 10 or 12;
R¹ is halogen;
m is 1; and
R² is H.

In some embodiments, the compound is

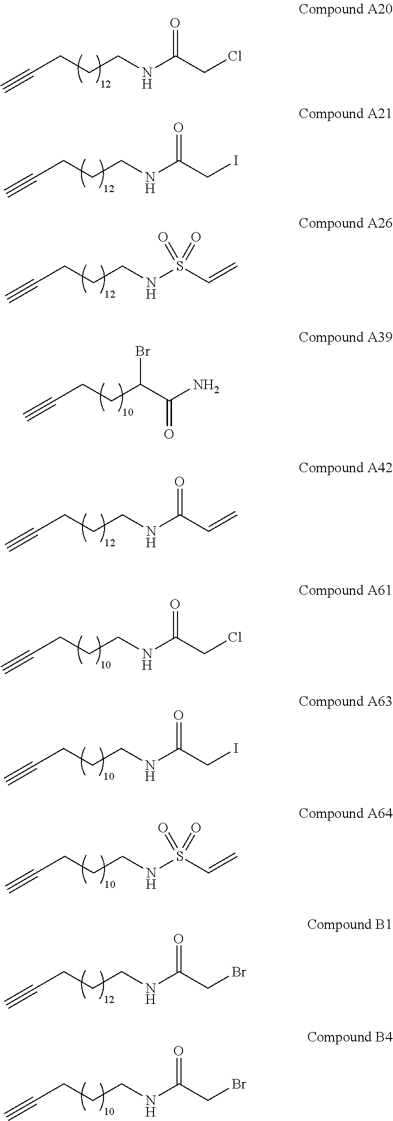

or a pharmaceutically acceptable salt of any of the foregoing.

In some embodiment, the compound is

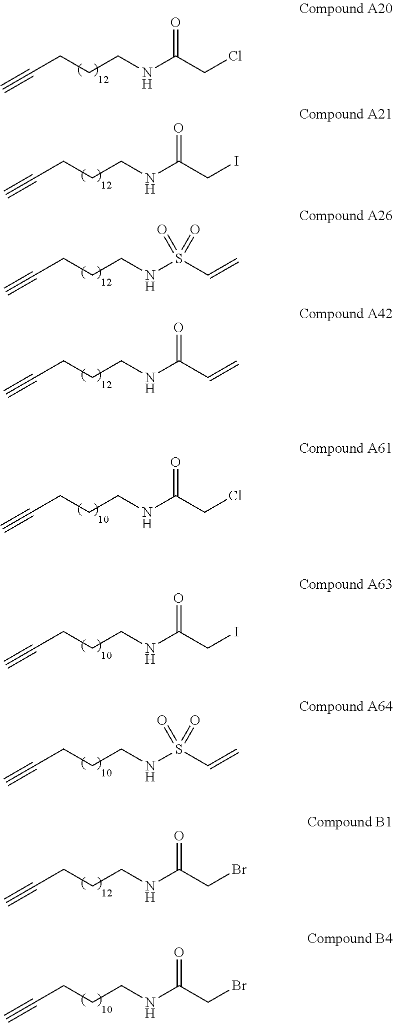

or a pharmaceutically acceptable salt of any of the foregoing.

In some embodiments, the compound is

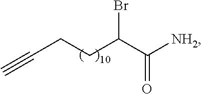

or a pharmaceutically acceptable salt thereof.

In some embodiment, the compound is

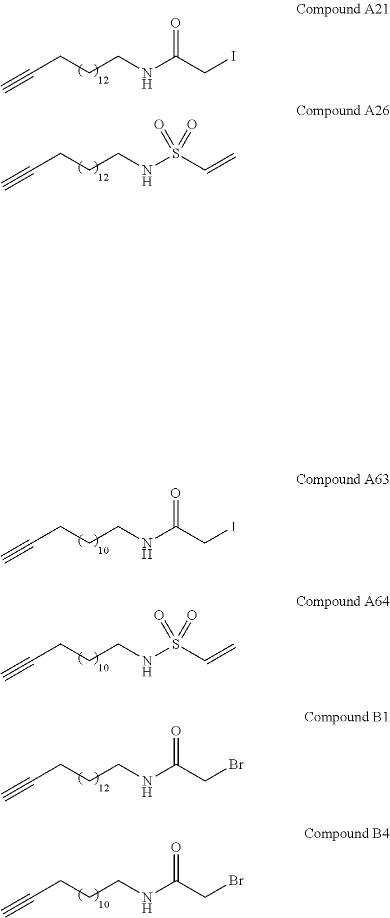

or a pharmaceutically acceptable salt of any of the foregoing.

In some embodiment, the compound is

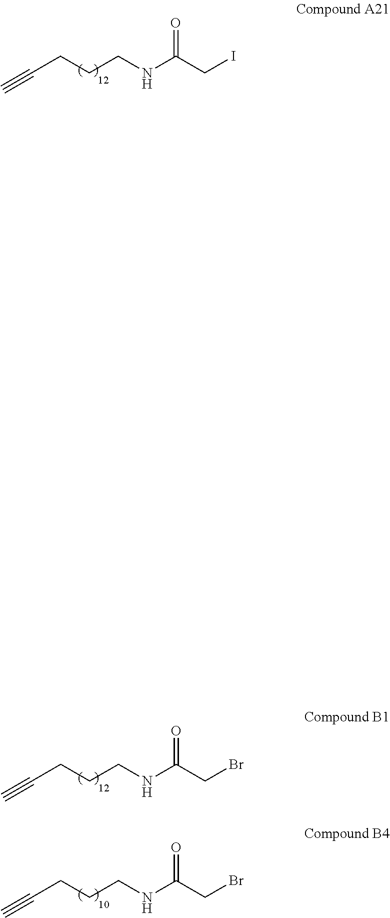

or a pharmaceutically acceptable salt of any of the foregoing.

In some embodiments, the compound is

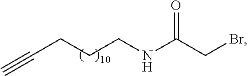

or a pharmaceutically acceptable salt thereof.

### Methods of Use

Provided herein is a method for identifying a hydrophobic pocket comprising
(a) contacting a cell, a cellular lysate, or a recombinant protein with a compound described herein forming Complex A;
(b) contacting Complex A with an indicator, forming Complex B;
(c) isolating Complex B; and
(d) identifying the hydrophobic pocket.

In some embodiments, the hydrophobic pocket is in proximity to a reactive nucleophile. In some embodiments, the reactive nucleophile is —SH. In some embodiments, the reactive nucleophile is a Cys sulfhydryl group.

In some embodiments, the indicator is DADPS biotin azide, Dde biotin azide, Dde biotin azide PLUS, Dde biotin picolyl azide, biotin picolyl azide, biotin azide, biotin azide PLUS, or PC azide.

Also provided herein is a method for identifying autolipidation, comprising:
(a) contacting a cell, a cellular lysate, or a recombinant protein with a compound described herein forming Complex A;
(b) contacting Complex A with a compound selected from the group consisting of DADPS biotin azide, Dde biotin azide, Dde biotin azide PLUS, Dde biotin picolyl azide, biotin picolyl azide, biotin azide, biotin azide PLUS, and PC azide, forming Complex B;
(c) isolating Complex B; and
(d) identifying the autolipidated protein.

Also provided herein is a method of identifying ligandable acylation sites comprising contacting a cell, a cellular lysate, or a recombinant protein with a compound described herein.

Also provided herein is a method of labeling cell lystates or tissue lysates comprising contacting a cellular lysate or a tissue lysate with a compound described herein.

Also provided herein is a method of identifying auto-palmitoylated proteins comprising contacting a cell, a cellular lysate, or a recombinant protein with a compound described herein.

### Definitions

As used herein, the term “Cₙ₋ₘ” indicates a range which includes the endpoints, wherein n and m are integers and indicate the number of carbons. Examples include C₁₋₄, C₁₋₆ and the like. Whenever the term is used, it is intended to describe each member included in the group, Cₙ through Cₘ as if each had been explicitly set forth. For example, the term C₁₋₆ is intended to describe each of the members C₁, C₂, C₃, C₄, C₅ and C₆.

As used herein, the term “alkyl” employed alone or in combination with other terms, refers to a saturated hydrocarbon group that may be straight-chain or branched. The term “Cₙ₋ₘ alkyl”, refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

As used herein, the term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl and the like

As used herein, the term “haloalkyl” refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1}halogen atoms, which may either be the same or different. In some embodiments, the halogen atoms are fluoro, chloro, bromo, or iodo atoms. In some embodiments, the haloalkyl group has 1 to 6 or 1 to 4 carbon atoms. Example haloalkyl groups include —CF₃, —CF₂CF₃, —CHF₂, —CCl₃, —CHCl₂, —CCl₂CCl₃, —CH₂Cl, —CH₂Br, —CH₂I, —CBr₃, —CH₂CH₂Br, —CH₂CH₂I, —CHBrCH₂Br, —CBr₂CH₃ and the like.

As used herein, “halo” or “halogen” refers to F, Cl, Br, or I. In some embodiments, a halo is I, Cl, or Br.

As used herein, the term “haloacetyl” refers to an acetyl group (—C(═O)CH₃), in which one or more hydrogen is substituted by a halogen, such as F, Cl, Br, or I. In one embodiment, the haloacetyl group is a chloroacetyl group (—C(═O)CH₂Cl). In another embodiment, the haloacetyl group is a bromoacetyl group (—C(═O)CH₂Br). In one embodiment, the haloacetyl is an iodoacetyl group (—C(═O)CH₂I).

The compounds described herein can be asymmetric (e.g., having one or more stereocenters). All stereoisomers, such as enantiomers and diastereoisomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereoisomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

In some embodiments, the compounds of the invention have the (R)-configuration. In other embodiments, the compounds have the (S)-configuration. In compounds with more than one chiral centers, each of the chiral centers in the compound may be independently (R) or (S), unless otherwise indicated.

The present invention also includes salts, particularly pharmaceutically acceptable salts, of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred.

### EXAMPLES

The following examples are illustrative and not intended to be limiting.

### Materials

All commercially available reagents were used without further purification. All solvents, such as ethyl acetate, DMSO, and dichloromethane (DCM), were ordered from Fisher Scientific and Sigma-Aldrich and used as received. Unless otherwise stated, all reactions were conducted under air. Analytical thin-layer chromatography (TLC) plates from Sigma were used to monitor reactions. Flash column chromatography was employed for purification and performed on silica gel (230-400 mesh). ¹H-NMR were recorded at 500 MHZ on JEOL spectrometer. ¹³C NMR were recorded at 125 MHz on JEOL spectrometer. The chemical shifts were determined with residual solvent as internal standard and reported in parts per million (ppm).

### Example 1: Intermediate 6a

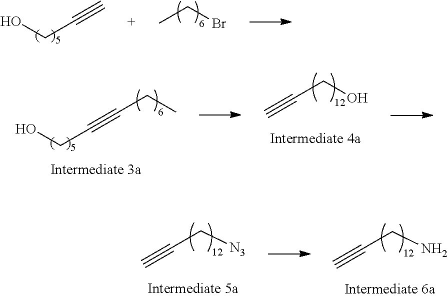

### Step 1: Tetradec-6-yn-1-ol (Intermediate 3a)

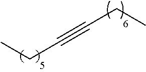

A solution of n-BuLi (5.1 mL, 12.83 mmol, 2.5 M in Hexane) was added dropwise to a mixture of hept-6-yne-ol (625.9 mg, 5.58 mmol), dry HMPA (11.2 mL) in THF (10 mL) at −15° C. The resulting solution was stirred at the same temperature for 0.5 h. A solution of 1-bromoheptane (500 mg, 2.79 mmol) in THF (4.7 mL) was added dropwise. The reaction was stirred at room temperature for another 3 h. After completion, the reaction was quenched with saturated NH₄Cl solution and extracted with ethyl acetate. The combined organic layer was washed with brine, dried over anhydrous Na₂SO₄ and concentrated in vacuo. The crude residue was purified using silica gel chromatography to give the desired Intermediate 3a as colorless oil (330 mg, 56%). ¹H NMR (500 MHz, Chloroform-d) δ 3.65 (q, J=6.5 Hz, 2H), 2.20-2.10 (m, 4H), 1.62-1.54 (m, 2H), 1.54-1.42 (m, 6H), 1.40-1.33 (m, 2H), 1.32-1.19 (m, 6H), 0.88 (t, J=6.9 Hz, 3H).

### Step 2: Tetradec-13-yn-1-ol (Intermediate 4a)

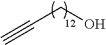

To NaH (7.9 equiv.), which was washed twice with dry hexane under Argon, was added 1,3-diaminopropane (DAP, 3.1 M). The mixture was stirred in an oil bath at 70° C. Evolution of gas was observed after 10 mins and the solution turned brown after 1 h. The flask was cooled down to rt, and a solution of Intermediate 3a (1.0 equiv, 330 mg, 1.57 mmol) in DAP (0.54 M) was added. The mixture was stirred at 55° C. overnight during which it turned black. The flask was cooled down to rt, carefully hydrolyzed with ice water, acidified with concentrated HCl, and extracted with EA. The combined organic layers were washed with brine, dried with Na₂SO₄. The solvent was removed under reduced pressure. The crude was purified with flash chromatographic column to give the desired Intermediate 4a as white solid (290 mg, 88%). ¹H NMR (500 MHz, Chloroform-d) δ 3.64 (t, J=6.6 Hz, 2H), 2.18 (td, J=7.1, 2.7 Hz, 2H), 1.94 (t, J=2.7 Hz, 1H), 1.60-1.48 (m, 4H), 1.41-1.21 (m, 16H).

### Step 3: 14-azidotetradec-1-yne (Intermediate 5a)

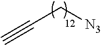

To a solution of Intermediate 4a (1.0 equiv, 160 mg, 0.761 mmol) in DCM (0.1 M) was added Et₃N (1.2 equiv), MsCl (1.5 equiv) at 0° C. The reaction was then stirred at rt. After reaction complete, the mixture was quenched with H₂O, diluted with DCM. The combined organic layers were washed with brine, dried with Na₂SO₄. The solvent was removed under reduced pressure. The obtained methane sulfonate was dissolved in DMF (0.1 M). The solution was added sodium azide (1.2 equiv) and stirred at 80° C. After reaction complete, the mixture was extracted with ethyl acetate and water, washed with brine, and dried with Na₂SO₄. The solvent was removed under reduced pressure. The crude was purified with flash chromatographic column to give the desired Intermediate 5a as colorless oil (140 mg, 89%). ¹H NMR (500 MHz, Chloroform-d) δ 3.26 (t, J=7.0 Hz, 2H), 2.18 (td, J=7.0, 2.4 Hz, 2H), 1.94 (t, J=3.4 Hz, 1H), 1.60 (p, J=7.2 Hz, 2H), 1.54-1.48 (m, 2H), 1.42-1.24 (m, 16H).

### Step 4: Tetradec-13-yn-1-amine (Intermediate 6a)

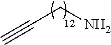

To a solution of Intermediate 5a (1 equiv, 310 mg, 1.5 mmol) in THF/H₂O (10:1, 0.15 M) was added PPh₃ (1.02 equiv). The reaction was stirred at rt. After reaction complete, the solvent was removed under reduced pressure. The crude material was purified using a flash chromatographic column to give the desired Intermediate 6a as white solid (260 mg, 85%). ¹H NMR (500 MHz, Chloroform-d) δ 2.68 (t, J=7.2 Hz, 4H), 2.17 (td, J=7.2, 2.7 Hz, 2H), 1.92 (t, J=2.7 Hz, 1H), 1.51 (p, J=7.2 Hz, 2H), 1.47-1.41 (m, 2H), 1.40-1.34 (m, 2H), 1.32-1.19 (m, 14H).

### Example 2: Intermediate 6b

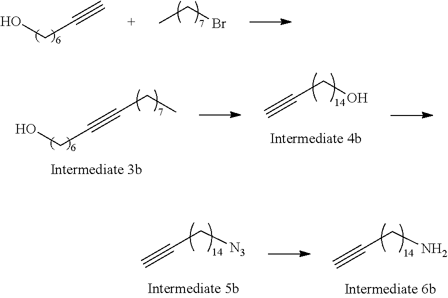

### Step 1: Hexadec-7-yn-1-ol (Intermediate 3b)

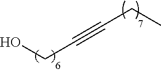

Hexadec-7-yn-1-ol (Intermediate 3b) was prepared according to the procedure outlined in Example 1 step 1, substituting hept-6-yne-ol with oct-7-yn-1-ol and 1-bromoheptane with 1-bromoheptane.

### Step 2: Hexadec-15-yn-1-ol (Intermediate 4b)

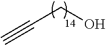

Hexadec-15-yn-1-ol (Intermediate 4b) was prepared according to the procedure outlined in Example 1 step 2, substituting Intermediate 3a with Intermediate 3b (1 g, 4.19 mmol), which resulted in Intermediate 4b as white solid (900 mg, 90%). ¹H NMR (500 MHz, Chloroform-d) δ 3.63 (t, J=6.6 Hz, 2H), 2.17 (td, J=7.1, 2.7 Hz, 2H), 1.93 (t, J=2.7 Hz, 1H), 1.60-1.48 (m, 4H), 1.41-1.21 (m, 20H).

### Step 3: 16-azidohexadec-1-yne (Intermediate 5b)

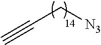

Intermediate 5b was prepared according to the procedure outlined in Example 1 step 3, substituting Intermediate 4a with Intermediate 4b (500 mg, 2.1 mmol), which resulted in Intermediate 5b as colorless oil (420 mg, 78%). ¹H NMR (500 MHz, Chloroform-d) δ 3.25 (t, J=7.0 Hz, 2H), 2.18 (td, J=7.1, 2.7 Hz, 2H), 1.94 (t, J=2.6 Hz, 1H), 1.60 (p, J=7.0 Hz, 2H), 1.55-1.49 (m, 2H), 1.42-1.24 (m, 20H).

### Step 4: Hexadec-15-yn-1-amine (Intermediate 6b)

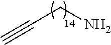

Intermediate 6b was prepared according to the procedure outlined in Example 1 step 4, substituting Intermediate 5a with Intermediate 5b (100 mg, 0.38 mmol), which resulted in Intermediate 6b as white solid (80 mg, 89%). ¹H NMR (500 MHz, Chloroform-d) δ 2.68 (t, J=7.1 Hz, 2H), 2.17 (td, J=7.2, 2.7 Hz, 2H), 1.93 (t, J=2.7 Hz, 3H), 1.51 (p, J=7.1 Hz, 2H), 1.47-1.34 (m, 4H), 1.33-1.20 (m, 18H).

### Example 3: 2-chloro-N-(hexadec-15-yn-1-yl)acetamide (Compound A20)

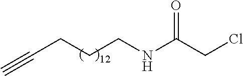

Intermediate 6b (60 mg, 0.25 mmol) was dissolved in DCM (0.5 M). To the solution was added triethylamine (2.0 equiv) and 2-chloroacetyl chloride (1.5 equiv) at 0° C. The mixture was stirred at room temperature for a further 2 h. The reaction was quenched with saturated NaHCO₃. The combined organic layers were washed with brine and dried with Na₂SO₄. The solvent was removed under reduced pressure. The crude residue was purified through a flash chromatograph column to obtain the desired Compound A20 as white solid (50 mg, 64%). ¹H NMR (500 MHz, Chloroform-c) δ 6.56 (br s, 1H), 4.05 (s, 2H), 3.30 (q, J=6.8 Hz, 2H), 2.18 (td, J=7.1, 2.6 Hz, 2H), 1.94 (d, J=2.6 Hz, 1H), 1.58-1.48 (m, 4H), 1.42-1.21 (m, 20H).

### Example 4: N-(hexadec-15-yn-1-yl)acrylamide (Compound A42)

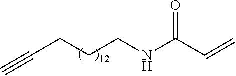

Compound A42 was prepared according to the procedure outlined in Example 3, substituting 2-chloroacetyl chloride with acryloyl chloride (20 μL, 0.252 mmol), which resulted in Compound A42 as white solid (22.6 mg, 46%). ¹H NMR (500 MHz, Chloroform-d) δ 6.27 (d, J=16.9 Hz, 1H), 6.08 (ddd, J=16.7, 10.2, 1.8 Hz, 1H), 5.63 (d, J=10.3 Hz, 1H), 5.56 (br s, 1H), 3.32 (q, J=7.6 Hz, 2H), 2.18 (ddt, J=7.2, 4.6, 2.5 Hz, 2H), 1.94 (q, J=2.5 Hz, 1H), 1.56-1.48 (m, 4H), 1.42-1.22 (m, 20H).

### Example 5: 2-bromo-N-(hexadec-15-yn-1-yl)acetamide (Compound B1)

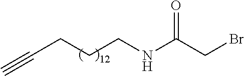

Compound B1 was prepared according to the procedure outlined in Example 3, substituting 2-chloroacetyl chloride with 2-bromoacetyl bromide (22 μL, 0.252 mmol), which resulted in Compound B1 as white solid (50 mg, 83%). ¹H NMR (500 MHz, Chloroform-d) δ 6.48 (br s, 1H), 3.88 (d, J=1.9 Hz, 2H), 3.32-3.24 (m, 2H), 2.18 (ft, J=7.0, 2.3 Hz, 2H), 1.94 (q, J=2.4 Hz, 1H), 1.59-1.47 (m, 4H), 1.43-1.19 (m, 20H).

### Example 6: 2-chloro-N-(tetradec-13-yn-1-yl)acetamide (Compound A61)

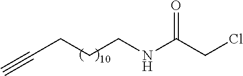

Compound A61 was prepared according to the procedure outlined in Example 3, substituting Intermediate 6b with Intermediate 6a, which resulted in Compound A61 as white solid (29.6 mg, 54%). ¹H NMR (500 MHz, Chloroform-d) δ 6.57 (br s, 1H), 4.04 (s, 2H), 3.29 (q, J=6.8 Hz, 2H), 2.17 (td, J=7.2, 2.6 Hz, 2H), 1.93 (t, J=2.7 Hz, 1H), 1.57-1.48 (m, 4H), 1.41-1.23 (m, 16H).

### Example 7: 2-bromo-N-(tetradec-13-yn-1-yl)acetamide (Compound B4)

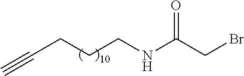

Compound B4 was prepared according to the procedure outlined in Example 3, substituting Intermediate 6b with Intermediate 6a and 2-chloroacetyl chloride with 2-bromoacetyl bromide, which resulted in Compound B4 as white solid (120 mg, 44%). ¹H NMR (500 MHz, Chloroform-d) δ 6.47 (br s, 1H), 3.88 (s, 2H), 3.28 (q, J=6.5 Hz, 2H), 2.18 (td, J=7.1, 2.6 Hz, 2H), 1.94 (t, J=2.7 Hz, 1H), 1.57-1.48 (m, 4H), 1.41-1.22 (m, 16H).

### Example 8: N-(hexadec-15-yn-1-yl)ethenesulfonamide (Compound A26)

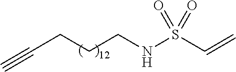

To a round flask containing Intermediate 6b (1.0 equiv, 80 mg, 0.34 mmol) and DMAP (0.1 equiv) was added DCM (0.2 M) and triethylamine (3.0 equiv). The mixture was then placed at 0° C., followed by addition of 2-chloroethane-1-sulfonyl chloride (1.4 equiv, 49 μL, 0.48 mmol) dropwise. After stirring at room temperature for further 3-4 h, the reaction was quenched with saturated NaHCO₃. The combined organic layers were washed with brine, dried with Na₂SO₄. The solvent was removed under reduced pressure. The crude residue was purified through flash chromatograph column to obtain Compound A26 as white solid (40 mg, 36%). ¹H NMR (500 MHz, Chloroform-d) δ 6.51 (dd, J=16.6, 9.9 Hz, 1H), 6.25 (d, J=16.5 Hz, 1H), 5.94 (d, J=9.9 Hz, 1H), 4.17 (t, J=6.2 Hz, 1H), 3.02 (q, J=6.8 Hz, 2H), 2.18 (td, J=7.1, 2.7 Hz, 2H), 1.94 (t, J=2.7 Hz, 1H), 1.60-1.48 (m, 4H), 1.42-1.22 (m, 20H).

### Example 9: N-(tetradec-13-yn-1-yl)ethenesulfonamide (Compound A64)

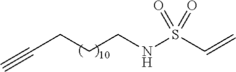

Compound A64 was prepared according to the procedure outlined in Example 8, substituting Intermediate 6b with Intermediate 6a, which resulted in Compound A64 as white solid (16 mg, 56%). ¹H NMR (500 MHz, Chloroform-d) δ 6.50 (dd, J=16.5, 10.0 Hz, 1H), 6.25 (d, J=16.6 Hz, 1H), 5.94 (d, J=9.9 Hz, 1H), 4.25 (br s, 1H), 3.01 (q, J=6.7 Hz, 2H), 2.18 (td, J=7.1, 2.7 Hz, 2H), 1.93 (t, J=3.0 Hz, 1H), 1.57-1.48 (m, 4H), 1.42-1.20 (m, 16H).

### Example 10: N-(hexadec-15-yn-1-yl)-2-iodoacetamide (Compound A21)

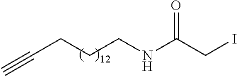

Compound A20 (1.0 equiv, 40 mg, 0.127 mmol) was dissolved in acetone (0.2 M). To the solution was added sodium iodide (2.5 equiv). The mixture was stirred at room temperature. The solvent was removed under reduced pressure. The crude residue was purified through a flash chromatograph column to obtain Compound A21 as yellow solid (50 mg, 97%). 1H NMR (500 MHz, Chloroform-d) δ 6.11 (br s, 1H), 3.69 (s, 2H), 3.25 (q, J=6.8 Hz, 2H), 2.17 (td, J=7.1, 2.7 Hz, 2H), 1.93 (t, J=2.7 Hz, 1H), 1.56-1.48 (m, 4H), 1.42-1.20 (m, 20H).

### Example 11: 2-iodo-N-(tetradec-13-yn-1-yl)acetamide (Compound A63)

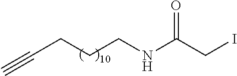

Compound A61 (1.0 equiv, 19.6 mg, 0.069 mmol) was dissolved in Acetone (0.2 M). To the solution was added sodium iodide (2.5 equiv). The mixture was stirred at room temperature. The solvent was removed under reduced pressure. The crude residue was purified through a flash chromatograph column to obtain Compound A63 as yellow solid (19.9 mg, 76%). ¹H NMR (500 MHz, Chloroform-d) δ 6.04 (br s, 1H), 3.69 (s, 2H), 3.26 (q, J=6.8 Hz, 2H), 2.18 (td, J=7.1, 2.6 Hz, 2H), 1.94 (t, J=2.6 Hz, 1H), 1.52 (p, J=7.1 Hz, 4H), 1.43-1.23 (m, 16H).

### Example 12: (Compound A39)

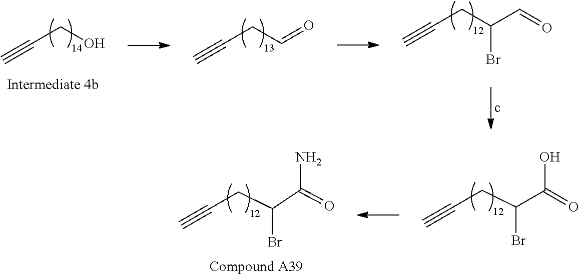

### Step 1: 15-Hexadecynal

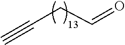

2-Iodobenzoic acid (1.76 g, 6.3 mmol) was dissolved in DMSO (6 mL). The solution was stirred at room temperature for 20 mins. Intermediate 4b (1 g, 4.2 mmol) was added. The reaction was stirred for another 3 h and was quenched with water. The mixture was then filtered, extracted with ether, washed with brine, and dried with Na₂SO₄. The solvent was removed under reduced pressure. The crude residue was purified through flash chromatograph column to obtain the desired 15-hexadecynal as colorless oil (350 mg, 35%). ¹H NMR (500 MHz, Chloroform-d) δ 9.76 (d, J=2.0 Hz, 1H), 2.42 (td, J=7.3, 1.9 Hz, 2H), 2.18 (td, J=7.1, 2.7 Hz, 2H), 1.94 (t, J=2.5 Hz, 1H), 1.67-1.59 (m, 2H), 1.55-1.48 (m, 2H), 1.44-1.22 (m, 18H).

### Step 2: 2-Bromohexadec-15-ynal

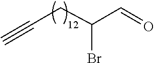

15-hexadecynal (150 mg, 0.636 mmol) was dissolved in 3 mL of DCM and was cooled to −10° C. Proline (87.6 mg, 0.763 mmol) was added and the mixture was stirred at −10° C. for 1 h. NBS (85 mg, 0.48 mmol) was then portion-wisely added, and temperature was kept below −5° C. After stirred at room temperature for further 1 h, the reaction was quenched with saturated NaHCO₃ and extracted with ethyl acetate. The combined organic layers were washed with brine, dried with Na₂SO₄. The solvent was removed under reduced pressure. The crude residue was purified through flash chromatograph column to obtain the desired 2-bromohexadec-15-ynal as colorless oil (110 mg, 55%). ¹H NMR (500 MHz, Chloroform-d) δ 9.43 (d, J=3.1 Hz, 1H), 4.21 (ddd, J=8.1, 6.2, 3.1 Hz, 1H), 2.18 (td, J=7.1, 2.6 Hz, 2H), 2.08-1.99 (m, 1H), 1.94 (t, J=2.6 Hz, 1H), 1.92-1.86 (m, 1H), 1.54-1.48 (m, 3H), 1.42-1.23 (m, 17H).

### Step 3: 2-Bromohexadec-15-ynoic acid

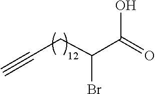

2-bromohexadec-15-ynal (110 mg, 0.35 mmol) was dissolved in DCM (2.3 mL) and DMF (5.8 mL). The solution was added pyridinium dichromate (261.3 mg, 1.272 mmol). The reaction was stirred at room temperature overnight and quenched with saturated NaHCO₃. The mixture was extracted with ethyl acetate. The combined organic layers were washed with brine and dried with Na₂SO₄. The solvent was removed under reduced pressure. The crude residue was purified through flash chromatograph column to obtain the desired 2-bromohexadec-15-ynoic acid as white solid (90 mg, 78%). ¹H NMR (500 MHz, Chloroform-d) δ 4.24 (t, J=7.3 Hz, 1H), 2.18 (td, J=7.1, 2.6 Hz, 2H), 2.13-2.04 (m, 1H), 2.03-1.96 (m, 1H), 1.94 (t, J=2.7 Hz, 1H), 1.56-1.47 (m, 3H), 1.42-1.23 (m, 17H).

### Step 4: 2-Bromohexadec-15-ynamide (Compound A39)

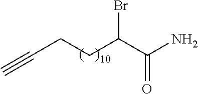

A solution of 2-bromohexadec-15-ynoic acid (50 mg, 0.151 mmol) in anhydrous DCM was added 1 drop of DMF and SOCl₂. The reaction was stirred at room temperature overnight. After removal of excess of SOCl₂ under vacuum, to the mixture was added slowly a cold solution of aqueous ammonium hydroxide and was allowed to warm slowly to room temperature with stirring. The mixture was then extracted with DCM, washed with brine, dried with Na₂SO₄. The crude residue was purified with flash chromatographic column to give the desired Compound A39 as white solid (14 mg, 28%). ¹H NMR (500 MHz, Chloroform-d) δ 6.31 (br s, 1H), 5.73 (br s, 1H), 4.29 (dd, J=8.3, 5.2 Hz, 1H), 2.18 (td, J=7.1, 2.6 Hz, 2H), 2.15-2.08 (m, 1H), 2.04-1.96 (m, 1H), 1.94 (t, J=2.6 Hz, 1H), 1.56-1.48 (m, 3H), 1.41-1.20 (m, 17H).

### Example A

### Recombinant Proteins

Recombinant PCNA (12131-H07B, Sino Biological), RAP1A (PRO-848, Prospec-Tany TechnoGene), RRAS2 (PRO-728, Prospec-Tany TechnoGene), IDH1 (ab113858, Abcam), IDH1R132H (14132, Cayman) are obtained from commercially available source. Recombinant TEAD2 protein were made in-house. All the recombinant proteins contain a His-tag.

### Labeling of TEAD2 by Covalent Probes In Vitro

500 ng of recombinant protein in 50 μL of MES buffer (50 mM, PH 6.4), was contacted with a solution of the covalent probe so that the final concentration of covalent probe in the reaction mixture is 10 μM of covalent probes. After incubation for 30 mins, 50 μL of sample mixture was treated with 5 μL of freshly prepared “click” mixture containing 100 μM TBTA (678937, Sigma-Aldrich), 1 mM TCEP (C4706, Sigma-Aldrich), 1 mM CuSO₄ (496130, Sigma-Aldrich), 100 μM Biotin-Azide (1167-5, Click Chemistry Tools) and incubated for another 1 h. To the samples was then added 11 μL of 6×SDS loading buffer (BP-111R, Boston Bio-Products) and the resultant mixture was denatured at 95° C. for 5 mins. SDS-PAGE was used to analyze the samples. Labeling signal was detected by streptavidin-HRP antibody (1:3000, S911, Invitrogen). The total protein level was detected by primary anti-His-tag antibody (1:10000, MA1-21315, Invitrogen) and secondary anti-mouse antibodies (1:5000, 7076S, Cell Signaling).

### Transfection

HEK293A cells was seed in 6 cm dishes overnight and transfected with plasmids using PEI reagent (1 μg/μL). Briefly, proteins of interest and PEI were diluted in serum-free DMEM medium in two tubes (DNA:PEI ratio=1:2). After standing still for 5 mins, the solutions were mixed well and allowed to stand unperturbed for an additional 20 mins. The mixture was then added to dishes directly.

### Labeling of Proteins by Covalent Probes in HEK293A Cells or HEK29A Lysate

For in-cell labeling, HEK293A cells with or without overexpression of proteins of interest were starved in DMEM medium with 10% dialyzed fetal bovine serum (DFBS) for 1 h and labeled using the covalent probes (10 μM) overnight. The cells were then washed and harvested using cold DPBS (14190250, Life Technologies). The cell pellets were isolated by centrifugation (500×g, 10 min) and lysed by TEA lysis buffer (50 mM TEA-HCl, pH 7.4, 150 mM NaCl, 1% Triton X-100, 0.2% SDS, 1×Protease inhibitor-EDTA free cocktail (05892791001, Roche), phosphatase inhibitor cocktail (P0044, Sigma-Aldrich)) on ice for 30 mins. The protein concentration was determined using Bio-Rad assay and adjusted to 1 mg/mL. For lysate labeling, HEK293A cells were grown in DMEM with 10% FBS. When confluence reached ˜90%, the cells were washed and harvested by cold DPBS. The cell pellets were isolated by centrifugation (500×g, 5 min) and resuspended with TEA lysis buffer (50 mM TEA-HCl, pH 7.4, 150 mM NaCl, 1% Triton X-100, IXProtease inhibitor-EDTA free cocktail, phosphatase inhibitor cocktail), incubated on ice for 15 mins, and then sonicated (7 s, 30% duty cycle, output setting=3). After centrifugation (17,000×g, 10 min), the supernatant is transferred to a clean tube. The protein concentration is determined using Bio-Rad assay and adjusted to 1 mg/mL. The lysate was then labeled with DMSO or probes (10 μM) at room temperature for 1 h with rotation. SDS was added to the lysate to a final concentration of 0.2% and the resultant solution retained for further use.

100 μL of labeled proteome was treated with 10 μL of freshly prepared “click” mixture containing 1 mM TBTA, 10 mM TCEP, 10 mM CuSO₄, 1 mM Biotin-Azide and incubated for 1 h at room temperature. The proteins were precipitated by chloroform/methanol/H₂O mixture. and redissolved with 2% SDS in 0.1% PBST. The solution was diluted with 0.1% PBST and incubated with prewashed streptavidin agarose beads (69203-3, E M D MILLIPORE). After rotation at room temperature for 2 h, the beads were then pelleted by centrifugation (500×g, 3 min) and washed with 0.2% SDS in PBS (3×1 mL). The bound proteins were eluted with a buffer containing 10 mM EDTA (pH 8.2) and 95% formamide and analyzed with SDS-PAGE. Anti-Myc (1:1000, 2278S, Cell Signaling), anti-pan-TEAD (1:1000, 13295, Cell Signaling), anti-HA (1:1000, 3724S, Cell Signaling) antibodies were used to detect Myc-TEAD1, pan-TEAD or HA-IDH1R132H, respectively. Secondary antibody was anti-rabbit (1:5000, 7074S, Cell Signaling).

### Mass Studies

To 1.5 mg of proteomes from cell labeling or lysate labeling as described above were added 150 μL fresh prepared “click” mixture of TBTA (75 μL, 2 mM in 1:4 DMSO: t-BuOH), CuSO₄ (30 μL, 50 mM in H₂O), TCEP (30 μL, 50 mM in H₂O) and Biotin-Azide (15 μL, 10 mM in DMSO). The samples were rotated at room temperature for 1 h. To these samples were then added MeOH (6 mL), CHCl₃ (1.5 mL) and H₂O (4.5 mL), and the resultant solution was mixed well. After centrifugation (17,000×g, 10 min), the aqueous layers were carefully aspirated leaving a protein disc between the organic and aqueous phases. The protein disc was washed with 6 mL of methanol and isolated by centrifugation (17,000×g, 10 min). The remaining protein pellet was collected and redissolved with 700 μL of 2% SDS in 0.1% PBST. The solution was diluted with 4.9 mL of 0.1% PBST and added 0.1% PBST-washed streptavidin agarose beads (150 μL, 1:1, sigma). After rotation at room temperature for 2 h, the beads were then pelleted by centrifugation (500×g, 5 min) and washed with 0.2% SDS in PBS (3×1 mL). The beads were transferred to a Protein Low-Binding tube and the immobilized proteins were digested by addition of urea (200 μL, 2.0 M in DPBS), CaCl₂) (2.0 μL, 100 mM in H₂O) and sequence grade porcine trypsin (2 μg; Promega). After digesting overnight at 37° C., the supernatant was transferred to a clean Protein Low-Binding, acidified with HCO₂H (formic acid, 16 μL) and stored at −20° C. until the analyzed by LC-MS/MS.

### Compound Treatment

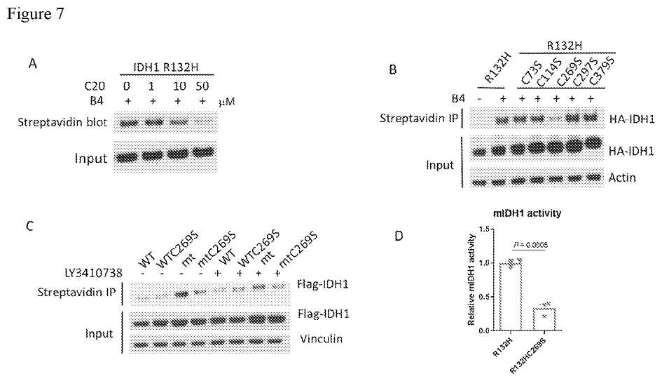

For in vitro treatment, the recombinant proteins were pretreated with compounds at indicated concentrations for 30 mins, followed by the labeling experiments. For in-cell treatment, HEK293A cells were pretreated with compounds under indicated concentrations for 8 h and then labeled with covalent probes for another 16 h. See FIGS. 7A and 7C. The structure of Compound C20 is shown below.

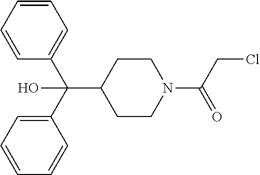 [SMILES: O=C(CCl)N1CCC(C(O)(c2ccccc2)c2ccccc2)CC1]

What is claimed is:

1. A compound according to Formula I: 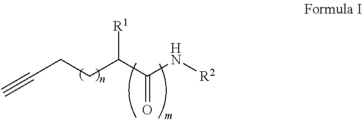
or a pharmaceutically acceptable salt thereof, wherein
n is 9, 10, 11, 12, 13, or 14;
R¹ is H or halogen;
m is 0 or 1; and
R² is H, —C(═O)—R^{A}, or —S(O)₂—R^{A};
wherein R^{A} is C₂₋₆ alkenyl or C₁₋₆ haloalkyl.

2. The compound of claim 1, or a pharmaceutically acceptable salt thereof, wherein n is 10 or 12.

3. The compound of claim 1 or 2, or a pharmaceutically acceptable salt thereof, wherein R¹ is H.

4. The compound of claim 1 or 2, or a pharmaceutically acceptable salt thereof, wherein R¹ is halogen.

5. The compound of claim 1 or 2, or a pharmaceutically acceptable salt thereof, wherein R¹ is Br.

6. The compound of any one of claims 1-5, or a pharmaceutically acceptable salt thereof, wherein m is 1.

7. The compound of claim 1 or 2, or a pharmaceutically acceptable salt thereof, wherein R¹ is Br and m is 1.

8. The compound of any one of claims 1-5, or a pharmaceutically acceptable salt thereof, wherein m is 0.

9. The compound of claim 1 or 2, or a pharmaceutically acceptable salt thereof, wherein R¹ is H and m is 0.

10. The compound of any one of claims 1-9, or a pharmaceutically acceptable salt thereof, wherein R² is C(═O)—R^{A}.

11. The compound of any one of claims 1-9, or a pharmaceutically acceptable salt thereof, wherein R² is —S(O)₂—R^{A}.

12. The compound of claim 10 or 11, or a pharmaceutically acceptable salt thereof, wherein R^{A} is alkenyl.

13. The compound of claim 10 or 11, or pharmaceutically acceptable salt thereof, wherein R^{A} is haloalkyl.

14. The compound of any one of claims 1-9, or a pharmaceutically acceptable salt thereof, wherein R² is H.

15. The compound of claim 1, or a pharmaceutically acceptable salt thereof, wherein
n is 10 or 12;
R¹ is H;
m is 0; and
R² is —C(═O)—R^{A}, or —S(O)₂—R^{A};
wherein R^{A} is C₂₋₆ alkenyl or C₁₋₆ haloalkyl.

16. The compound of claim 1, or a pharmaceutically acceptable salt thereof, wherein
n is 10 or 12;
R¹ is halogen;
m is 1; and
R² is H.

17. The compound of claim 1, wherein the compound is 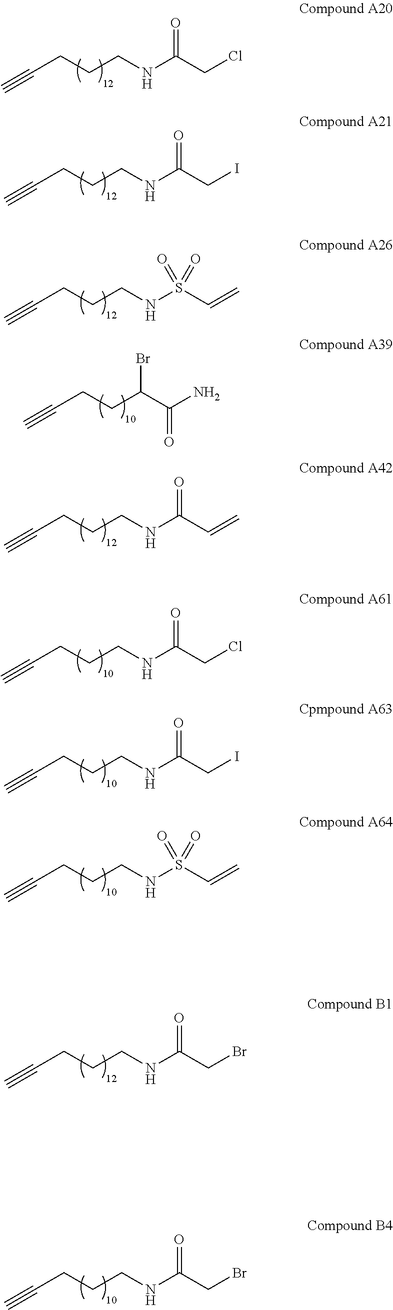
or a pharmaceutically acceptable salt of any of the foregoing.

18. A method for identifying autolipidation, comprising:
(a) contacting a cell, a cellular lysate, or a recombinant protein with a compound of any one of claims 1-17, or a pharmaceutically acceptable salt thereof, forming Complex A;
(b) contacting Complex A with a compound selected from the group consisting of DADPS biotin azide, Dde biotin azide, Dde biotin azide PLUS, Dde biotin picolyl azide, biotin picolyl azide, biotin azide, biotin azide PLUS, and PC azide, forming Complex B;
(c) isolating Complex B; and
(d) identifying the autolipidated protein.

19. A method of identifying ligandable acylation sites comprising contacting a cell, a cellular lysate, or a recombinant protein with a compound of any one of claims 1-17, or a pharmaceutically acceptable salt thereof.

20. A method of labeling cell lystates or tissue lysates comprising contacting a cellular lysate or a tissue lysate with a compound of any one of claims 1-17, or a pharmaceutically acceptable salt thereof.

21. A method of identifying auto-palmitoylated proteins comprising contacting a cell, a cellular lysate, or a recombinant protein with a compound of any one of claims 1-17, or a pharmaceutically acceptable salt thereof.
